(19) United States
(12) Patent Application Publication
(10) Pub. No.: US 2019/0000790 A1
(43) Pub. Date: Jan. 3, 2019

(54) PPAR-GAMMA ACTIVATORS AND THEIR THERAPEUTICAL USAGES

(71) Applicant: THERIAC BIOMEDICALE INC., MONT-ROYAL (CA)

(72) Inventor: Tony ANTKALY, MONTREAL (CA)

(21) Appl. No.: 15/780,904

(22) PCT Filed: Nov. 30, 2016
(86) PCT No.: PCT/CA2016/051405

§ 371 Date: Jun. 1, 2018

Related U.S. Application Data
(60) Provisional application No. 62/261,625, filed on Dec. 1, 2015.

(51) Int. Cl. A61K 31/222; A61K 31/192

(52) U.S. Cl. CPC A61K 31/222; A61K 31/192

(57) ABSTRACT

The invention relates to a composition for induction of activity of a nuclear receptor PPARy in a subject in need thereof, which comprises at least one of benzoate or a synergistic combination of benzoate and phenylacetate in association with a pharmaceutical carrier.

### BACKGROUND

### (a) Field

The subject matter disclosed generally relates to a novel composition of PPARγ activators including benzoate (BNZ) and phenylacetate (PAA) and their therapeutical usages.

### (b) Related Prior Art

Benzoate (BNZ) and phenylacetate (PAA) were individually studied for possible bioactivities in reducing inflammation, cancer growth and alleviating symptoms in animal models of experimental neurological disorders, namely multiple sclerosis, ALS, Huntington's disease and encephalopathy.

The clinical use of PAA and BNZ in lowering plasma ammonium levels in patients with lethal hyperammonemia is a hallmark of their therapeutic action (1, 2). In certain metabolic diseases resulting from defects in urea cycle enzymes, ammonium, which cannot be converted to urea, accumulates to a toxic level that can be lethal. A drug combination of PAA and BNZ is particularly useful to treat patients with congenital errors of metabolism of the urea cycle enzyme, thus preventing complications such as encephalopathy (1, 2). In fact, PAA, through mitochondrial conjugation with glutamine, results in the formation of phenylacetylglutamine compound. Similarly, BNZ combines with glycine forming benzoylglycine (hippuric acid). These two non-toxic compounds, phenylacetyglutamine and benzoylglycine are easily eliminated in the urine. Enns et al. (2) reported the results of a 25-year clinical study, using a drug consisting of a combination of PAA and BNZ, to treat patients with urea cycle disorders, demonstrating an overall survival rate of 84%.

PAA and phenylbutyrate (PBA), the parent compound from which it is metabolized, both display important bioactivities other than those cited above. Chemically, PAA and PBA belong to a group of aromatic fatty acids having a stable phenyl ring. These compounds were proven useful in the treatment of several diseases, including, sickle cell anemia (3), amyotrophic lateral sclerosis (4), Huntington's disease (5), neuronal inflammatory conditions (6) and cancer (7). Initially, PAA was discovered as a plant hormone that regulates cell growth (8). It has been extensively studied in the past two decades as an anti-cancer agent and cellular differentiating compound in laboratory settings and clinical trials. In fact, PAA inhibits the growth of several cancer cell types of different lineages and, in some instances, it promotes their differentiation to a non-cancerous phenotype. Of interest are the effects of PAA and PBA on gliomas and neuroblastomas, originally thought to be mediated by the inhibition of protein prenylation as well as cholesterol and fatty acid biosynthesis (5). Studies have demonstrated that PAA and PBA inhibit the growth of several neoplastic cell types, including breast cancer, prostate cancer, colon cancer and thyroid carcinoma. These anti-cancer actions of PAA and PBA have prompted the initiation of several clinical trials since these compounds display little toxicity if any (4, 5, 7).

The exact mechanisms underlying these physiological and pharmacological effects of BNZ, PAA and its butyrate metabolites are not completely known, but regulatory effects of butyrate derivatives have involved inhibition of histone deacetylation, which modulates chromatin conformation and regulation of nuclear receptor gene expression (9). Since PAA and PBA can potentiate the action of other hormones, such as estrogens and retinoids, in regulating cancer cell growth, we hyothezized that they might regulate the expression of members of the nuclear receptor family (which includes steroids, retinoids, and peroxisome proliferator-activated receptor). In fact, our previous work have shown that retinoids inhibit Kaposi's sarcoma cancer cell growth by activating the retinoic acid receptor (10).

We investigated whether PAA or BZN could regulate members of the nuclear receptors. We found that they each activate the peroxisome proliferator-activated receptor γ (PPARγ) which belongs to the steroid/nuclear receptor family of ligand-activated transcription factors (11). However to date, PAA and BNZ were never combined to show a synergistic effect of activators of PPARγ and BNZ alone was never shown to activate PPARγ.

### SUMMARY

According to an embodiment, there is provided a composition for induction of activity of a nuclear receptor PPARγ in a subject in need thereof, which comprises at least one of:
benzoate;
a synergistic combination of benzoate and phenylacetate;

in association with a pharmaceutical carrier.

The use of the composition, wherein the induction of activity of a nuclear receptor PPARγ improves symptoms of at least one of Inflammation and pain (osteoarthritis, rheumatoid arthritis), pain, autoimmune diseases (Lupus erythematous), neurodegenerative inflammatory diseases (Multiple Sclerosis, Parkinson disease, Alzheimer, ALS, Huntington), anti-cancer, diabetes type 2, to replace PPARγ agonists in other metabolic diseases.

According to another embodiment, there is provided use of a synergistic combination of benzoate and phenylacetate in association with a pharmaceutical carrier for inducing activity of a nuclear receptor PPARγ in a subject in need thereof.

The use of this combination, wherein the inducing activity of a nuclear receptor PPARγ improves symptoms of at least one of inflammation and pain (osteoarthritis, rheumatoid arthritis), psoriatic arthritis, juvenile arthritis, ankylosing spondylitis, gout, pain, autoimmune diseases (Lupus erythematous), neurodegenerative inflammatory diseases (Multiple Sclerosis, Parkingson disease, Alzheimer, ALS, Huntington), anti-cancer, diabetes type 2, to replace PPARγ agonists in other metabolic diseases.

According to another embodiment, there is provided a method of inducing activity of a nuclear receptor PPARγ in a subject in need thereof, which comprises administering to the subject a synergistic combination of benzoate and phenylacetate in association with a pharmaceutical carrier.

The inducing activity of a nuclear receptor PPARγ improves symptoms of at least one of Inflammation and pain (osteoarthritis, rheumatoid arthritis), psoriatic arthritis, juvenile arthritis, ankylosing spondylitis, gout, pain, autoimmune diseases (Lupus erythematous), neurodegenerative inflammatory diseases (Multiple Sclerosis, Parkinson disease, Alzheimer, ALS, Huntington), anti-cancer, diabetes type 2, to replace PPARγ agonists in other metabolic diseases.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1A, 1B illustrate a transcriptional activation of PPARγ by BNZ and PAA. Luciferase assay was performed in 293 cells transfected with Gal4-hPPARγ and UAStkLuc reporter construct and treated for 16 h with respective compounds. Data are expressed as fold change ±SEM compared to untreated cells set at 1.0.*, P<0.05; **, P<0.001.

FIG. 1C illustrates the additive effect of SB and PAA on transcriptional activity of PPARγ. Luciferase assay was performed in 293 cells transfected with Gal4-hPPARγ and UAStkLuc reporter construct and treated 10 mM of each compound for 16 h. Data are expressed as fold change ±SEM compared to untreated cells set at 1.0.*, P<0.001.

FIG. 2A, 2B, 2C illustrate prospective, randomized, double blinded testing the functional efficacy of a mixture of BZN and PAA against a positive (Pregabalin and Carprofen) and a negative (Placebo) control in the rat osteoarthritis (OA) instability surgical (cranial cruciate ligament transection+medial meniscus destabilization). (N=12 per group). All procedures were normalized. Abbrev. SWB (static weigh bearing), RMTS (response to mechanical temporal summation).

FIGS. 3A, 3B, 3C illustrate the pressure application method (PAM) demonstrates effect of HIP-002 (the code name for the mixture of BZN and PAA) on allodynia. Osteoarthritis was surgically induced by sectioning the medial meniscus and anterior cruciate ligament of the right knee seven days before start of treatment. Pain thresholds were evaluated by the pressure application method and expressed as grams of pressure applied to the right paw at the moment of withdrawal from the apparatus. Higher values suggest higher tolerance for pain. All data are presented as mean±SEM. (3A) Time course of PAM experiment. Baseline measures were taken one day before start of treatment (day −1). Rats then received daily subcutaneous injections of placebo (open circles), positive control (5 mg/kg of carprofen, an NSAID and 30 mg/kg of pregabalin, a potent analgesic (black circles), HIP-002 low dose (benzoate and phenylacetate respectively: 32.9 and 2.7 mg/kg, upward triangles) or HIP-002 high dose (molecules 1 and 2 respectively: 107.1 and 11.2 mg/kg, downward triangles). Pressure application measures were taken weekly (days 7, 14, 21, 28 and 35). Two-way repeated measures ANOVA detected significant effects of time (P<0.0001), treatment (P=0.0005) and time x treatment interaction (P=0.0007). Holm-Sidak post hoc comparisons against placebo group found statistically significant differences at days 14 and 28 of treatment, denoted by asterisks and arrows. (*) P<0.05; (**) P<0.01; (***) P<0.001. (3B) Bar graph showing values at day 14. Note a clear difference between groups when compared to placebo. Open circles indicate each individual values. P Values of Holm-Sidak post hoc comparisons are denoted on graph. (3C) Bar graph showing values at day 28 as in B. Although the differences are small, both HIP-002 treated groups are still significantly higher than the placebo group.

FIG. 4 illustrates the reduction of substance P levels in the spinal cord of osteoarthritic rats following 56 days of treatment with HIP-002. Rats were euthanized and the spinal cords were dissected, homogenized and processed for HPLC/MS-MS measurement of substance P levels. Substance P level is known as a marker for pain. Data are expressed in femtomoles per milligram of tissue (fmol/mg). Bars represent mean±SEM. Treatment regimens are presented under each bar, namely: placebo (white), positive control (5 mg/kg of carprofen and 30 mg/kg of pregabalin, dark grey) or HIP-002 (benzoate and phenylacetate respectively: 35.8 and 1.8 mg/kg, blue). Open circles indicate each individual measurement. One-way ANOVA indicated a significant effect of treatment (P=0.0022). P values obtained with Holm-Sidak post hoc comparisons against placebo group are denoted on the graph.

FIGS. 5A, 5B illustrates histopathological analysis of knees of osteoarthritic rats. Osteoarthritis was surgically induced by sectioning the medial meniscus and anterior cruciate ligament of the right knee. Treatment started seven days after surgery. Rats then received subcutaneous injections of placebo, positive control (5 mg/kg of carprofen and 30 mg/kg of pregabalin) or BZN+PAA (code named HIP-002). Following 36 days of treatment all rats were euthanized and knees were dissected, sectioned and stained for histopathological analysis. In general, the operated knee displayed diffuse chronic inflammation of the joint capsule and mild granular basophilic material in the joint cavity which was observed in all groups. Minimal to mild diffuse chronic inflammation of the joint capsule and minimal granular basophilic material with cell debris/eosinophilic material in the joint cavity were observed in the animals of all three groups where osteoarthritis was induced by surgical method but at a higher level in placebo animals. Summary of right (operated) knee microscopic findings are presented in Table 1 below. The highest severity of the findings was seen in Placebo (Control) group, where no positive treatment and/or treatment by HIP-002 was performed. Severity of the diffuse chronic inflammation was lower in the Molecule HIP-002 group (7 minimal and 3 mild) when compared with Positive Control group (4 minimal and 6 mild) and Placebo Control group (3 minimal and 7 mild).

FIG. 6 illustrates BZN+PAA (HIP-002) treatment of T cells derived from healthy volunteer (HV) and Systemic Lupus Erythematous (SLE) patients.

### DETAILED DESCRIPTION

The combination of benzoate and phenylacetate induce the activity of the nuclear receptor PPARγ. This transcription factor is a member of the steroid receptor superfamily and master regulator of lipid metabolism, inflammation and a key target for insulin-regulating drugs.

Furthermore, the usefulness of concomitant treatment of benzoate and phenylacetate in the treatment of osteoarthritis is shown in a rodent model of osteoarthritis.

Reporter Gene Bioassay of PPAR Transcriptional Activity

Human embryonic kidney 293 cells were seeded in DMEM supplemented with 5% charcoal dextran-treated FBS and transfected with Gal4 DBD fusion of PPAR isoforms and UAStkLuc reporter as described (12). Cells were then treated for 16 hrs and harvested for luciferase activity. Luciferase values were normalized for transfection efficiency to β-galactosidase activity and expressed as relative fold response compared to untreated cells. Data are derived from at least four independent experiments performed in triplicates.

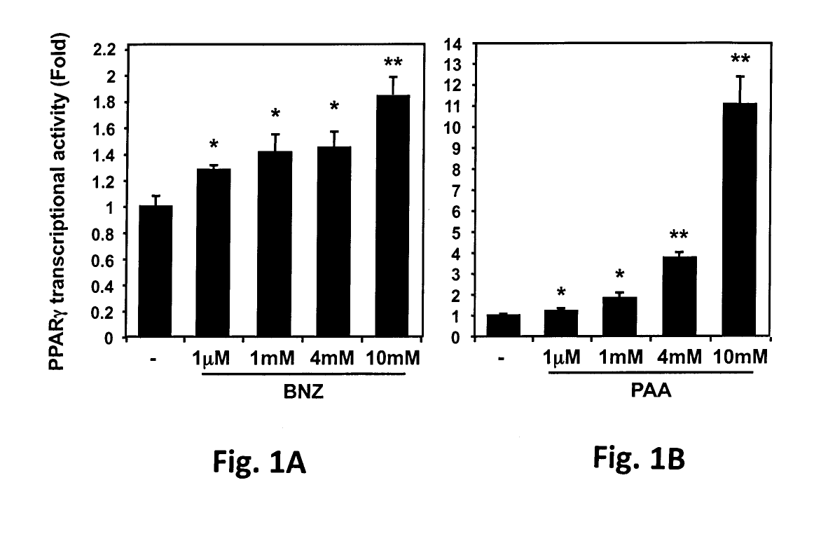

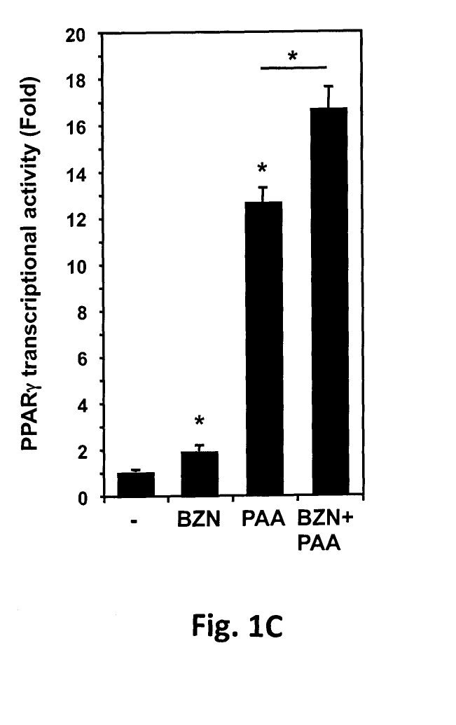

Human embryonic kidney 293 cells were transfected with a luciferase reporter gene construct under the control of a Gal4-DNA binding upstream-activating sequence (UAStkLuc) in the presence of an expression plasmid encoding Gal4 DNA-binding domain fusion to human PPARγ. Therefore, we tested their respective action on PPARγ activity and found that both compounds were significantly potent in increasing PPARγ activity (FIGS. 1 A and B). More importantly, when these two molecules were tested in combination, the resulting stimulation of PPARγ activity is much higher that each molecule alone reaching up to 18 fold that of placebo control (FIG. 1C). It is widely accepted that such a significant level of increased activity of a transcription factor will effectively result in synergistic induction of target gene expression These results identify PAA and BNZ as PPAR activators.

Effect of a Combined Use of PAA and BZN in Inflammation and Pain Perception

A validated animal model of osteoarthritis (13) was used to test the effect of daily concomitant administration of PAA and BZN on inflammation and arthritis induced pain. Adult female ovariectomized rats were rendered osteoarthtitic (OA) after surgical cranial cruciate ligament transection and medial meniscus destabilization of the hind knee. After a rest period, animals were divided into three groups and injected daily, subcutaneously. The first group (N=12) which served as positive control, received a mixture of Pregbalin also known as Lyrica™ (a gabapentinoid with analgesic action) and Carprofen, an NSAID (non-steroidal anti-inflammatory). The second group (N=12) received a mixture of PAA and BZN in normal saline (experimental group). The third group (N=12) was the placebo control and was injected with normal saline.

Animals were tested at the indicated times using three methods to assess response. The first method is static weight bearing (measuring the force put by the rat on both hind limbs)(21). The second method measured tactile hypersensitivity (measuring the withdrawal threshold to von Frey stimulation)(22). A third method measured RTMS (Response Mechanical Temporal Summation) consisting of applying a mechanical stimulus at regular frequency (30 stimulations) and a constant pressure (2 Newton), then tolerance time was recorded (IITC Life Science, Woodland Hills, Calif.)(23). The less the pain, the more the animal will tolerate the mechanical stress. This test is known to assess the efficacy of analgesic molecules by characterizing the phenomenon of central sensitization (13).

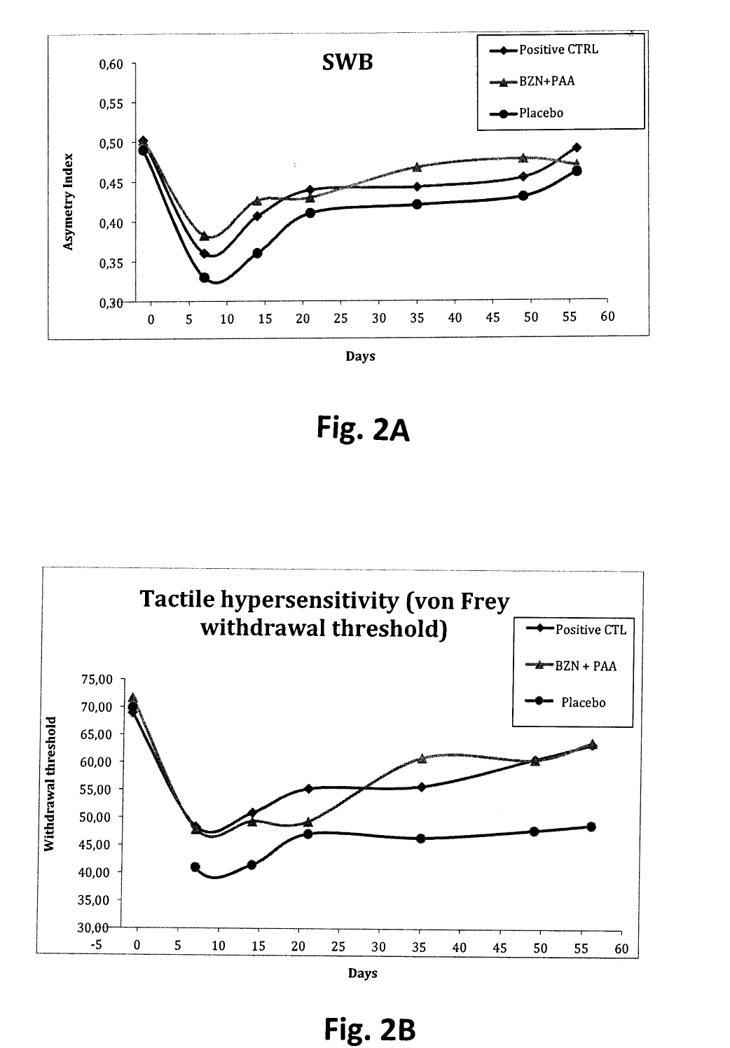

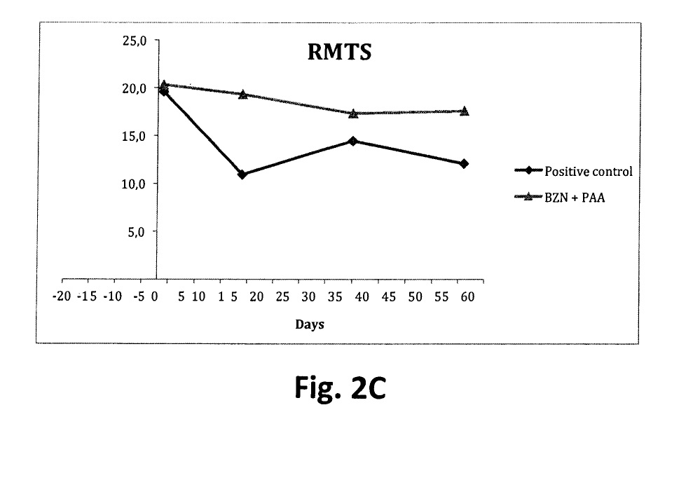

The asymmetry index as outcome for the force put by the rat on both hind limbs was calculated, demonstrating high reproducibility between groups at Baseline (day 0). Over the two months of follow-up post-surgical OA induction, a linear mixed model for repeated measures was used with time as intra-subject factor and groups as inter-subjects factor, and the baseline value as covariate. There was a significant time (P<0.0001), and time x group (P=0.05) effect, but no group effect (P=0.2584). A priori contrasts were tested with Benjamini-Hochberg correction for multiple comparisons. They were significant only for group 2 (PAA+BZN) over Placebo at Day 35 and Day 49 (P<0.04). Therefore, in this study, the kinetics analysis is demonstrating a significant treatment effect of group 2 over placebo in the chronic phase of the model, i.e. Day 35 and Day 49 (FIG. 2). There was no significant difference at any time-point between group 2 and the positive control group. This suggests that PAA and BZN was as effective as a recognized standard in this osteoarthritis model. Similar to previous evaluations using a surgical or chemical OA induction model in the rat, the asymmetry index is coming back to baseline after an abrupt alteration.

This positive and interesting effect of PAA+BZN over Placebo in the kinetics analysis was associated with a beneficial effect observed on tactile hypersensitivity. At the difference of kinetics, in this surgical OA rat model, the installed tactile hypersensitivity was not regressing in the negative control (Placebo) group. After Day 21, the withdrawal threshold to von Frey stimulation was stable over time for the Placebo group (FIG. 2B). Using the same statistical model, we observed an excellent reproducibility of assessment at baseline, and over the whole period of follow-up a significant time (P<0.0001), and group (P=0.0086) effect, as well as a significant time x group interaction (P=0.045). The a priori contrasts were significant between Placebo and Positive Control at Day 56 (P=0.0012), and between Placebo and PAA+BZN at Day 35 (P=0.004) and Day 56 (P=0.0028). Moreover, at any time-point there was any significant difference between PAA+BZN and the Positive Control (FIG. 2B). The beneficial effect of PAA+BZN on tactile hypersensitivity was further supported in experiments using the pressure application method.

Osteoarthritis was surgically induced by sectioning the medial meniscus and anterior cruciate ligament of the right knee one day before start of treatment. Baseline measurements were taken one day before surgery (day −1). Rats then received daily subcutaneous injections of placebo (open circles), positive control (5 mg/kg of carprofen and 30 mg/kg of pregabalin, black circles) or low dose HIP-002 (molecules 1 and 2 respectively: 35.8 and 1.8 mg/kg, upward triangles). Measurements were taken at 7, 21, and 56 days after beginning of treatment. Tactile allodynia was evaluated by applying a nylon filament coupled to a force transducer (electronic von Frey apparatus) to the plantar surface of the ipsilateral paw. Force was gradually increased and the threshold to observe a typical paw withdrawal response, in grams, was recorded. Smaller values indicate presence of tactile allodynia. Two-way repeated measures ANOVA detected significant effects of time (P<0.0001) and treatment (P=0.0105). Holm-Sidak post hoc comparisons against placebo group found statistically significant differences 56 days after treatment. Static weight bearing experiment. Impairment of operated leg as evaluated by measuring the percentage of total body weight supported by said leg when rats are put on a standing posture. Values closest to 50% suggest no impairment. Lower values indicate greater impairment. Two-way repeated measures ANOVA detected significant effects of time (P<0.0001) and treatment (P=0.0076). Holm-Sidak post hoc comparisons against placebo group found statistically significant differences at days 7 and 21. All data are presented as mean±SEM. (*) P<0.05; (**) P<0.01.

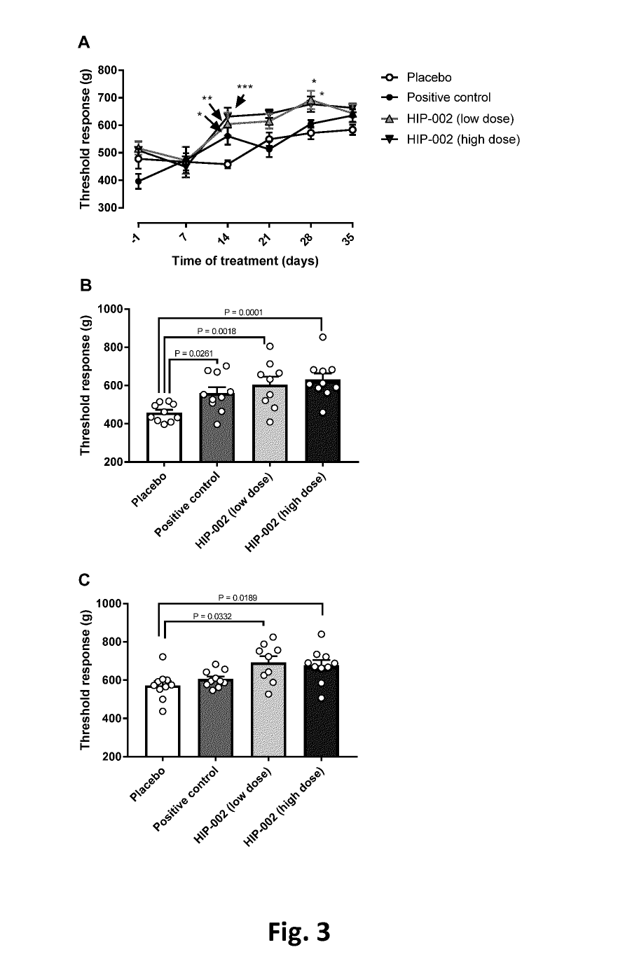

In the case of RMTS evaluation, (FIG. 2C) we observed that PAA+BZN treatment is even better that the positive control (p<0.05) at day 14 and 55. This situation is similar to experiments using the pressure application method (FIG. 3) in which PAA+BZN were found to be more potent than the positive control indicating that those animals treated with PAA+BZN have higher tolerance for pain that those treated with positive control.

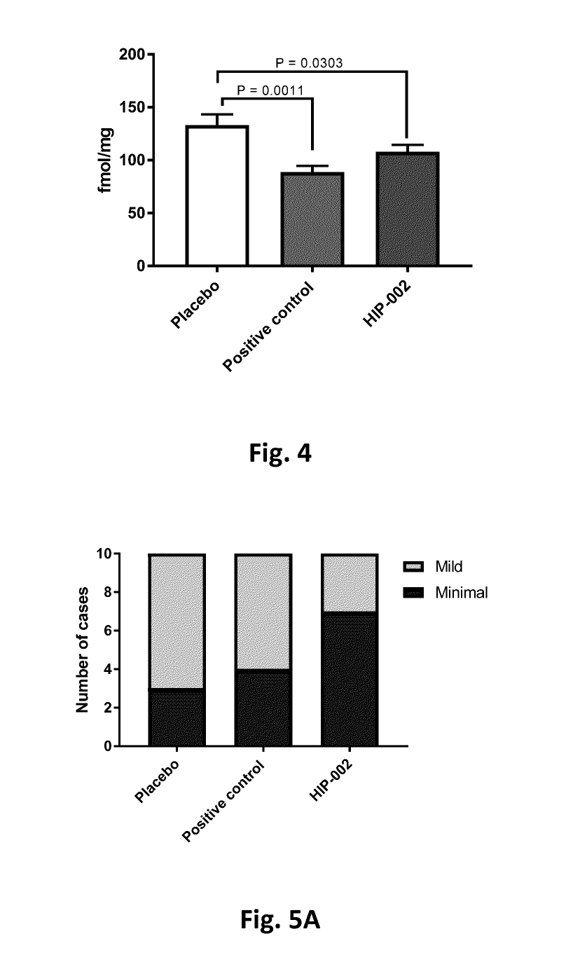

Overall, these evaluations are suggesting an anti-neuropathic effect of PAA+BZN. This is indeed confirmed by the measurement of spinal neuropeptides. Using HPLC/MS-MS we quantified the different tachykinins released in the spinal chord after OA induction. We observe that in a Placebo group, OA induction is associated with an increased release in Substance P, and endomorphin. The release of these three neuropeptides is reduced in the Positive Control group treated with Pregabalin and Carprofen. Similarly, the same anti-neuropathic effects of BZN+PAA is demonstrated with the measurement of these neuropeptides which are reduced (FIG. 4). In addition, other relevant neuropeptides and PPAR levels are currently being measured in other tissues autopsied from all experimental animals namely, brain (hypothalamus, brain minus hypothalamus, pituitary, spinal cord, PBMC). Furthermore, we observe changes in serum levels of inflammation bio-markers such as cytokines IL-1, 11-2, 11-6 and TNF. Currently, 17 major cytokines involved in inflammation are being measured using a multiplex immunoassay.

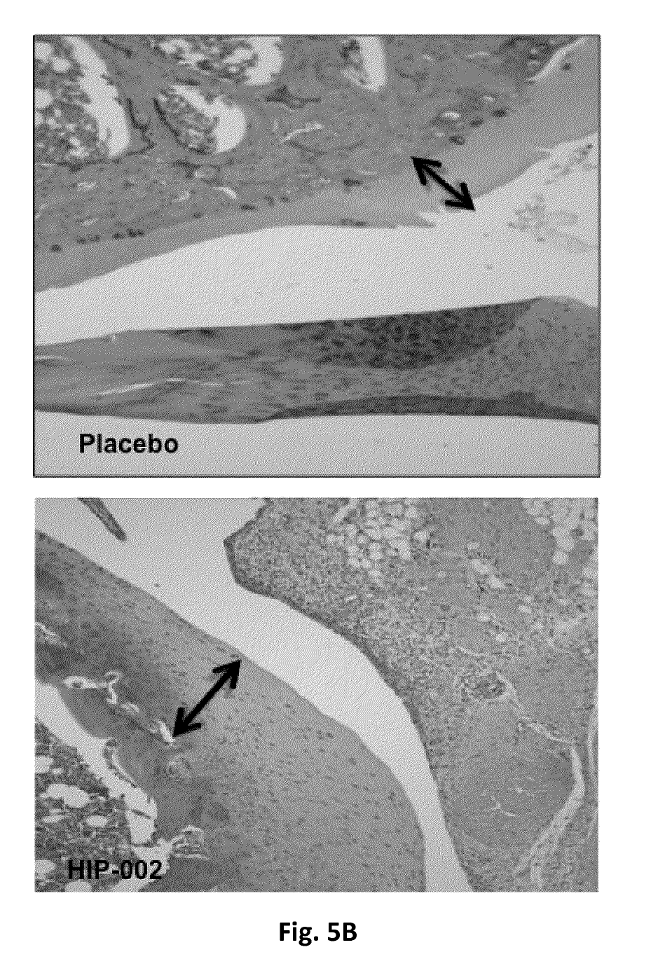

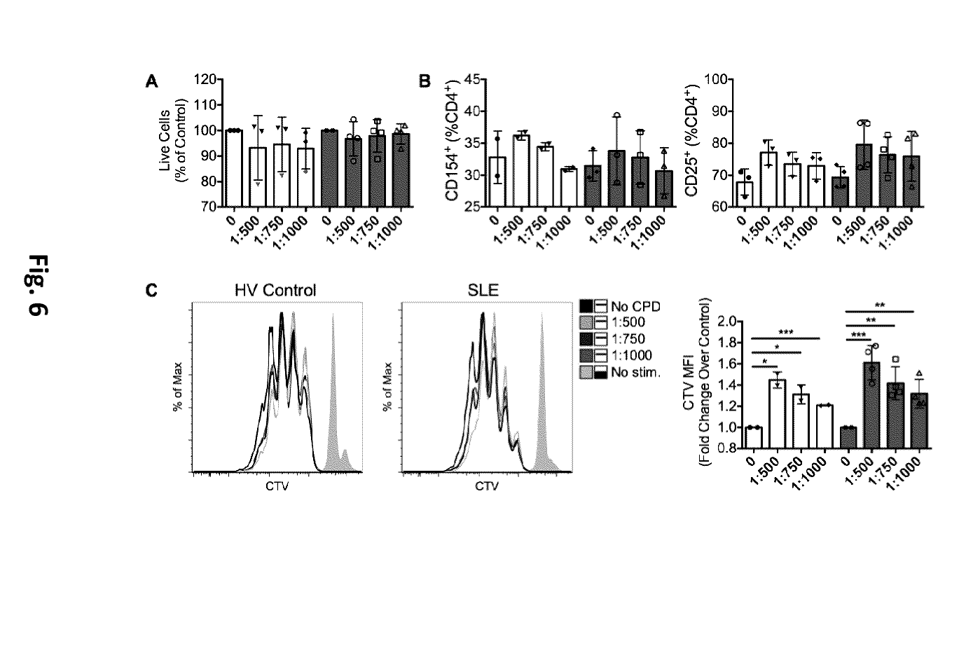

The histological differences were also examined from tissue sections prepared the affected knees of all experimental groups. As shown in FIG. 5A, BZN+PAA seem more beneficial than placebo and even better than the positive control in reducing joint inflammation as well as loss of the cartilage layer and death of chondrocytes (FIG. 5B, see micrographs). Note severe depletion of the cartilage area (double head arrows) and its chondrocytes (FIG. 5B). Note also the basophilic material representing tissue debris in knee's joint cavity of the placebo (FIG. 5B).

TABLE 1
Microscopic findings of the right (treated) knee

| | Positive Control | | Placebo | Molecule HIP-002 | |
|---|---|---|---|---|---|
| | Pre- | Car- | (Con- | Phenyl- | Na |
| Group | gabalin | profen | trol) | acetate | Benzoate |
| Dose Level (mg/kg/day) | 30 | 5 | 0 | 12.5 | 125 |

| | | | |
|---|---|---|---|
| Number of animals | 10 | 10 | 10 |
| Chronic inflammation | 10 | 10 | 10 |
| (diffuse) | | | |
| minimal | 4 | 3 | 7 |
| mild | 6 | 7 | 3 |
| Granular basophilic | 0 | 2 | 1 |
| material | | | |

Next, we examined if BZN+PAA can improve immune function of T lymphocytes in Systemic Lupus Erythematosus (SLE). Preliminary data is shown in FIG. 6, support that BZN has beneficial effect on the growth and survival of these cells which contribute to improve the immune response. Dilutions effects of the BZN+PAA drug (HIP-002) are shown. (FIG. 6A). CD4+ T cells were stimulated with α-CD3/CD28 beads for 4 (red) or 5 (black) days and cytotoxicity determined by Live/Dead staining. (FIG. 6B) CD4+ T cells were stimulated as in (FIG. 6A) for 5 days, and the surface expression of CD154 and CD25 measured by FACS analysis. (FIG. 6C) CD4+ T cells were stimulated as in (FIG. 6A), and proliferation measured by dilution of the CellTrace Violet dye. White bars: HV; n=2-3. Gray bars: SLE patients; n=3-4. * p<0.05, ** p<0.01, *** p<0.0001.

Given PPAR's central role in regulating essential aspects of whole body lipid, glucose and energy metabolism, the activation of PPARγ by PAA, BNZ and/or related compounds, might represent an effective strategy for treating metabolic disorders, inflammation and carcinogenesis. Current PPAR gamma agonists were shown to display significant toxicity in patients resulting in their restrictions on their clinical use. On the other hand, PAA and BNZ were shown to have no significant toxicity.

The present invention will be more readily understood by referring to the following examples which are given to illustrate the invention rather than to limit its scope.

### Example 1

Indications for the Mixture of PAA & BZN in Humans and Animals
Inflammation and pain including but not limited to osteoarthritis, rheumatoid arthritis, atherosclerosis, periodontitis, hay fever
Psoriatic arthritis
Juvenile arthritis
Ankylosing spondylitis
Gout
Pain
Autoimmune diseases including but not limited to Addison's disease, Agammaglobulinemia, Alopecia areata, Amyloidosis, Ankylosing spondylitis, Anti-GBM/Anti-TBM nephritis, Antiphospholipid syndrome (APS), Autoimmune hepatitis, Autoimmune inner ear disease (AIED), Axonal & neuronal neuropathy (AMAN), Behcet's disease, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid/benign mucosal pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, Huntington disease, IgA Nephropathy, IgG4-related sclerosing disease, Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lupus erythematous, Lyme disease chronic, Meniere's disease, Microscopic polyangiitis (MPA), Mixed connective tissue disease (MCTD), Mooren's ulcer, Mucha-Habermann disease, Multiple sclerosis (MS), Myasthenia gravis, Myositis, Narcolepsy, Neuromyelitis optica, Neutropenia, Ocular cicatricial pemphigoid, Optic neuritis, Palindromic rheumatism (PR), PANDAS (Pediatric Autoimmune Neuropsychiatric Disorders Associated with Streptococcus), Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome (polyneuropathy, organomegaly, endocrinopathy, monoclonal gammopathy, skin changes), Polyarteritis nodosa, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Reiter's syndrome, Relapsing polychondritis, Restless legs syndrome (RLS), Retroperitoneal fibrosis, Rheumatic fever, Rheumatoid arthritis (RA), Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Wegener's granulomatosis (now termed Granulomatosis with Polyangiitis (GPA)
Neurodegenerative inflammatory diseases including but not limited to Multiple Sclerosis, Parkinson disease, Alzheimer, ALS or Lou Gehrig's disease, Huntington
Cancer
Diabetes type 2
To Replace PPAR gamma agonists in other metabolic diseases
To regulate gene expression by boosting histone deacetylase inhibition.

While preferred embodiments have been described above and illustrated in the accompanying drawings, it will be evident to those skilled in the art that modifications may be made without departing from this disclosure. Such modifications are considered as possible variants comprised in the scope of the disclosure.

### REFERENCE LIST

1. Brusilow S W, et al. (1984) Treatment of episodic hyperammonemia in children with inborn errors of urea synthesis. N. Engl. J. Med. 310(25):1630-1634.
2. Enns G M, et al. (2007) Survival after treatment with phenylacetate and benzoate for urea-cycle disorders. N. Engl. J. Med. 356(22):2282-2292.
3. Resar L M, et al. (2002) Induction of fetal hemoglobin synthesis in children with sickle cell anemia on low-dose oral sodium phenylbutyrate therapy. J. Pediatr. Hematol. Oncol. 24(9):737-741.
4. Cudkowicz M E, et al. (2009) Phase 2 study of sodium phenylbutyrate in ALS. Amyotroph. Lateral. Scler. 10(2):99-106.
5. Phuphanich S, et al. (2005) Oral sodium phenylbutyrate in patients with recurrent malignant gliomas: a dose escalation and pharmacologic study. Neuro. Oncol. 7(2):177-182.
6. Brahmachari S, Jana A, & Pahan K (2009) Sodium benzoate, a metabolite of cinnamon and a food additive, reduces microglial and astroglial inflammatory responses. J. Immunol. 183(9):5917-5927.
7. Camacho L H, et al. (2007) Phase I dose escalation clinical trial of phenylbutyrate sodium administered twice daily to patients with advanced solid tumors. Invest. New Drugs 25(2):131-138.
8. Wightman F & Lighty D C (1982) Identification of phenylacetic acid as a natural auxin in the shoots of higher plants. physiol plant 55:17-24.
9. Merzvinskyte R, Treigyte G, Savickiene J, Magnusson K E, & Navakauskiene R (2006) Effects of histone deacetylase inhibitors, sodium phenyl butyrate and vitamin B3, in combination with retinoic acid on granulocytic differentiation of human promyelocytic leukemia HL-60 cells. Ann. N. Y. Acad. Sci. 1091:356-367.
10. Guo W X, Gill P S, & Antakly T (1995) Inhibition of AIDS-Kaposi's sarcoma cell proliferation following retinoic acid receptor activation. Cancer Res. 55:823-829.
11. Ahmadian M, et al. (2013) PPARgamma signaling and metabolism: the good, the bad and the future. Nat. Med. 19(5):557-566.
12. Avallone R, et al. (2006) A growth hormone-releasing peptide that binds scavenger receptor CD36 and ghrelin receptor up-regulates sterol transporters and cholesterol efflux in macrophages through a peroxisome proliferator-activated receptor gamma-dependent pathway. Mol. Endocrinol. 20(12):3165-3178.
13. Guillot M, et al. (2014) Evoked temporal summation in cats to highlight central sensitization related to osteoarthritis-associated chronic pain: a preliminary study. PLoS One 9(5):e97347.

1. A composition for induction of activity of a nuclear receptor PPARγ in a subject in need thereof, which comprises at least one of:
benzoate;
a synergistic combination of benzoate and phenylacetate
in association with a pharmaceutical carrier.

2. Use of the composition of claim 1, wherein said induction of activity of a nuclear receptor PPARγ improves symptoms of at least one of inflammation, pain, autoimmune diseases, neurodegenerative inflammatory diseases, cancer, diabetes type 2, and other metabolic diseases to replace PPARγ agonists.

3. The use of claim 2, wherein inflammation is chosen from osteoarthritis, rheumatoid arthritis, atherosclerosis, periodontitis, hay fever; autoimmune diseases is chosen from Addison's disease, Agammaglobulinemia, Alopecia areata, Amyloidosis, Ankylosing spondylitis, Anti-GBM/Anti-TBM nephritis, Antiphospholipid syndrome (APS), Autoimmune hepatitis, Autoimmune inner ear disease (AIED), Axonal & neuronal neuropathy (AMAN), Behcet's disease, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid/benign mucosal pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, Huntington disease, IgA Nephropathy, IgG4-related sclerosing disease, Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lupus erythematous, Lyme disease chronic, Meniere's disease, Microscopic polyangiitis (MPA), Mixed connective tissue disease (MCTD), Mooren's ulcer, Mucha-Habermann disease, Multiple sclerosis (MS), Myasthenia gravis, Myositis, Narcolepsy, Neuromyelitis optica, Neutropenia, Ocular cicatricial pemphigoid, Optic neuritis, Palindromic rheumatism (PR), PANDAS (Pediatric Autoimmune Neuropsychiatric Disorders Associated with Streptococcus), Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome (polyneuropathy, organomegaly, endocrinopathy, monoclonal gammopathy, skin changes), Polyarteritis nodosa, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Reiter's syndrome, Relapsing polychondritis, Restless legs syndrome (RLS), Retroperitoneal fibrosis, Rheumatic fever, Rheumatoid arthritis (RA), Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Wegener's granulomatosis (now termed Granulomatosis with Polyangiitis (GPA); and neurodegenerative inflammatory diseases is chosen from Multiple Sclerosis, Parkinson disease, Alzheimer, ALS or Lou Gehrig's disease, Huntington.

4. Use of a synergistic combination of benzoate and phenylacetate in association with a pharmaceutical carrier for inducing activity of a nuclear receptor PPARγ in a subject in need thereof.

5. The use of claim 4, wherein said inducing activity of a nuclear receptor PPARγ improves symptoms of at least one of inflammation, pain, autoimmune diseases, neurodegenerative inflammatory diseases, cancer, diabetes type 2, and other metabolic diseases to replace PPARγ agonists.

6. The use of claim 5, wherein inflammation is chosen from osteoarthritis, rheumatoid arthritis, atherosclerosis, periodontitis, hay fever; autoimmune diseases is chosen from Addison's disease, Agammaglobulinemia, Alopecia areata, Amyloidosis, Ankylosing spondylitis, Anti-GBM/Anti-TBM nephritis, Antiphospholipid syndrome (APS), Autoimmune hepatitis, Autoimmune inner ear disease (AIED), Axonal & neuronal neuropathy (AMAN), Behcet's disease, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid/benign mucosal pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, Huntington disease, IgA Nephropathy, IgG4-related sclerosing disease, Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lupus erythematous, Lyme disease chronic, Meniere's disease, Microscopic polyangiitis (MPA), Mixed connective tissue disease (MCTD), Mooren's ulcer, Mucha-Habermann disease, Multiple sclerosis (MS), Myasthenia gravis, Myositis, Narcolepsy, Neuromyelitis optica, Neutropenia, Ocular cicatricial pemphigoid, Optic neuritis, Palindromic rheumatism (PR), PANDAS (Pediatric Autoimmune Neuropsychiatric Disorders Associated with Streptococcus), Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome (polyneuropathy, organomegaly, endocrinopathy, monoclonal gammopathy, skin changes), Polyarteritis nodosa, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Reiter's syndrome, Relapsing polychondritis, Restless legs syndrome (RLS), Retroperitoneal fibrosis, Rheumatic fever, Rheumatoid arthritis (RA), Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Wegener's granulomatosis (now termed Granulomatosis with Polyangiitis (GPA); and neurodegenerative inflammatory diseases is chosen from Multiple Sclerosis, Parkinson disease, Alzheimer, ALS or Lou Gehrig's disease, Huntington.

7. A method of inducing activity of a nuclear receptor PPARγ in a subject in need thereof, which comprises administering to said subject a synergistic combination of benzoate and phenylacetate in association with a pharmaceutical carrier.

8. The method of claim 5, wherein said inducing activity of a nuclear receptor PPARγ improves symptoms of at least one of inflammation, pain, autoimmune diseases, neurodegenerative inflammatory diseases, cancer, diabetes type 2, and other metabolic diseases to replace PPARγ agonists.

9. The method of claim 8, wherein inflammation is chosen from osteoarthritis, rheumatoid arthritis, atherosclerosis, periodontitis, hay fever; autoimmune diseases is chosen from Addison's disease, Agammaglobulinemia, Alopecia areata, Amyloidosis, Ankylosing spondylitis, Anti-GBM/Anti-TBM nephritis, Antiphospholipid syndrome (APS), Autoimmune hepatitis, Autoimmune inner ear disease (AIED), Axonal & neuronal neuropathy (AMAN), Behcet's disease, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid/benign mucosal pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, Huntington disease, IgA Nephropathy, IgG4-related sclerosing disease, Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lupus erythematous, Lyme disease chronic, Meniere's disease, Microscopic polyangiitis (MPA), Mixed connective tissue disease (MCTD), Mooren's ulcer, Mucha-Habermann disease, Multiple sclerosis (MS), Myasthenia gravis, Myositis, Narcolepsy, Neuromyelitis optica, Neutropenia, Ocular cicatricial pemphigoid, Optic neuritis, Palindromic rheumatism (PR), PANDAS (Pediatric Autoimmune Neuropsychiatric Disorders Associated with Streptococcus), Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome (polyneuropathy, organomegaly, endocrinopathy, monoclonal gammopathy, skin changes), Polyarteritis nodosa, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Reiter's syndrome, Relapsing polychondritis, Restless legs syndrome (RLS), Retroperitoneal fibrosis, Rheumatic fever, Rheumatoid arthritis (RA), Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Wegener's granulomatosis (now termed Granulomatosis with Polyangiitis (GPA); and neurodegenerative inflammatory diseases is chosen from Multiple Sclerosis, Parkinson disease, Alzheimer, ALS or Lou Gehrig's disease, Huntington.
